(19) United States
(12) Patent Application Publication
(10) Pub. No.: US 2023/0355287 A1
(43) Pub. Date: Nov. 9, 2023

(54) BONE FRACTURE FIXATION DEVICE

(71) Applicant: Stabiiz Orthopaedics, Inc., Exton, PA (US)

(72) Inventors: Douglas Cerynik, Wynnewood, PA (US); Anjan Shah, Tampa, FL (US)

(73) Assignee: Stabiliz Orthopaedics, Inc., Exton, PA (US)

(21) Appl. No.: 18/312,592

(22) Filed: May 5, 2023

Related U.S. Application Data
(60) Provisional application No. 63/338,568, filed on May 5, 2022.

(51) Int. Cl. A61B 17/86

(52) U.S. Cl. CPC A61B 17/8685; A61B 17/8605

(57) ABSTRACT

A device for securing a fractured bone together includes a main screw configured to be driven through a bone fracture, where the main screw engages a removable screw head. Another device secures a fractured bone together and includes a main screw driven through a bone fracture where the main screw includes a slot configured to engage a cross screw therethrough, where the slot includes a system that prevents lateral movement of the main screw relative to the cross screw. Yet another includes a main screw with a main portion and a movable portion, where the movable portion moves within the main portion, and where one of the main portion or movable portion includes threads configured to engage bone. And another device includes a main peg with a hook that: extends from a channel through the peg configured to engage bone.

### BACKGROUND

Bone fracture fixation technology is focused primarily on hip fractures and more specifically, on femoral neck fractures. There are at least four main problems with current technologies:
Femoral neck shortening
Rotation of femoral head
Screw cutout
Avascular necrosis (AVN)

The historical way to fix these fractures is with cannulated screws. The problem with cannulated screws is that they may fail up to 30% of the time. To address these complications, variations in screw size, quantities and configurations have been implemented, but each of the prior solutions continues to struggle to overcome the problems mentioned herein.

FIGS. 1 and 2 show a classic neck fracture and how 3 screws can fail. FIG. 3 shows an attempt to use three screws in a particular configuration to attempt to overcome the problems in FIGS. 1 and 2.

Alternately, FIG. 4A shows another way to fix these breaks is using a dynamic hip screw (DHS). Implanting a DHS requires a longer surgical time, greater blood loss, a large incision, and the removal of a substantial amount of healthy bone which may lead to AVN and screw cutout

More recently, the femoral neck system (FNS) shown in FIG. 4B tries to prevent the problems specified above but still leads to considerable complication rates. Failures from using this system include shortening, rotation, and cutout

Alternatively, some surgeons may elect to perform joint replacement surgery (arthroplasty) in place of repairing femoral neck fractures because of the known complications with existing implants.

### SUMMARY OF THE EMBODIMENTS

The benefits of the device and method herein and also in U.S. Pat. No. 11,213,334 and US Publication 20220192723 (herein incorporated by reference as if fully set forth herein) reduce all of the above challenges and attempts to preserve femoral neck length, control (limit) rotation of the femoral head, prevent screw cutout, limit volume of hardware inside the bone to reduce risk of AVN.

A device for securing a fractured bone together includes a main screw configured to be driven through a bone fracture, where the main screw engages a removable screw head. Another device secures a fractured bone together and includes a main screw driven through a bone fracture where the main screw includes a slot configured to engage a cross screw therethrough, where the slot includes a system that prevents lateral movement of the main screw relative to the cross screw. Yet another includes a main screw with a main portion and a movable portion, where the movable portion moves within the main portion, and where one of the main portion or movable portion includes threads configured to engage bone. And another device includes a main peg with a hook that: extends from a channel through the peg configured to engage bone.

### BRIEF DESCRIPTION OF THE DRAWINGS

FIGS. 1-3, 4A, and 4B show prior art attempts to address femoral neck fractures.

FIGS. 5A and 5B show a threaded end cap head on the main screw.

FIGS. 6A and 6B show a washer or articulating head that conforms to the lateral bone surface.

FIGS. 7A and 7B show another variation on the embodiment in FIGS. 5A and 5B.

FIGS. 8A-8C show a main screw ring/shelf located in its open slot that engages the cross screw.

FIGS. 9A-9C show a polymer (resorbing or non-resorbing) insert that snaps into the main screw clearance slot.

FIGS. 10A-10D show a main screw slot with a portion occupied by a polymer that engages the cross screw.

FIGS. 11A and 11B show a resorbing core that threads/snaps into the interior of main screw.

FIGS. 12A-12D show a set screw that threads/snaps into an interior of the main screw.

FIGS. 13A-D show extendible hooks that extend from an end of the main screw to prevent its rotation and inadvertent withdrawal.

FIGS. 14A-14D show the hook extending through a channel that extends along the main screw length to an opening.

FIGS. 15A to 15C show a telescoping main screw.

### DETAILED DESCRIPTION OF THE EMBODIMENTS

The invention herein describes different approaches for joining together a femoral neck fracture or any bone fragment fracture using various screws. Figures having like numbers show different views and variations of each approach, though many of the approaches are merely variations on more prominent themes.

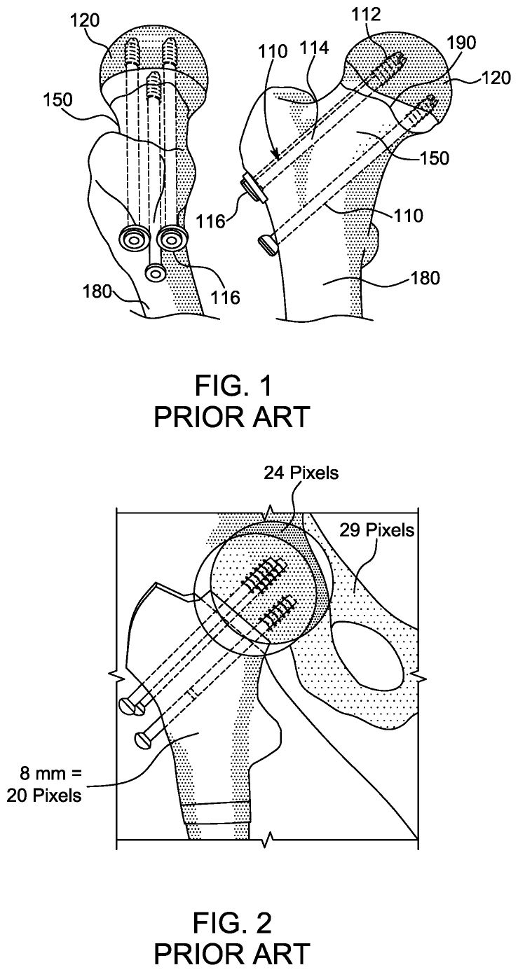

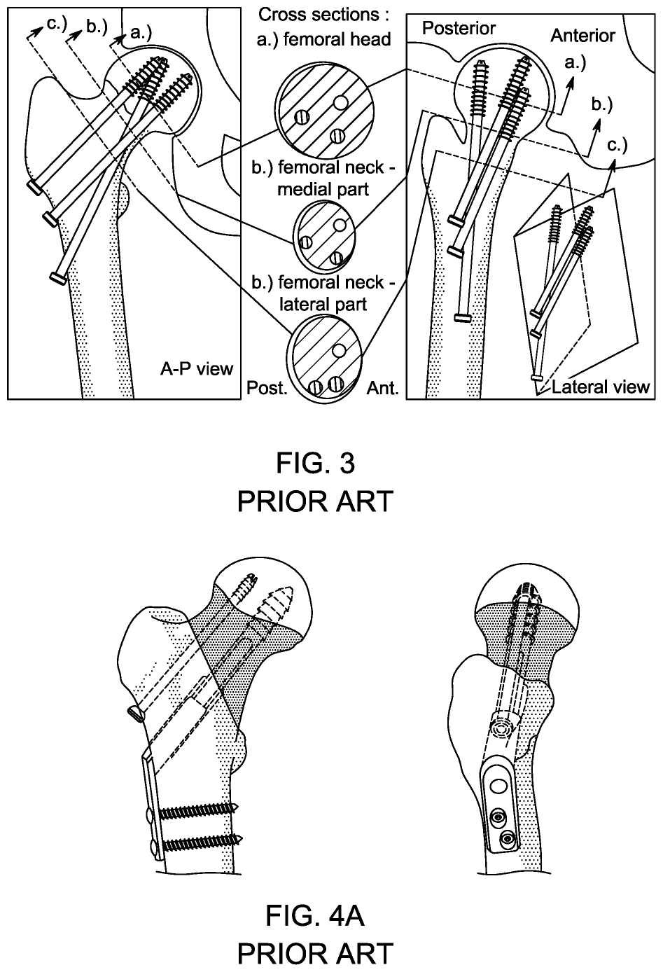

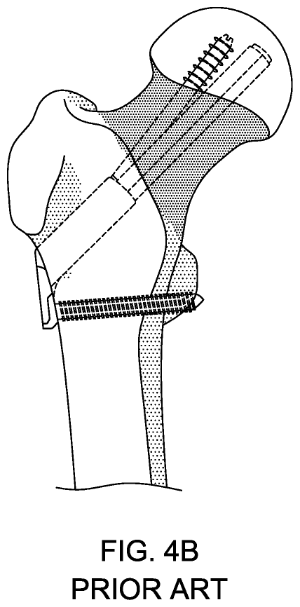

In the figures, the common elements are the bone head 120, the bone neck 150, and bone shaft 180. Some figures may show the bone fracture 190, while others may not show bone details. Other common elements include bone screws (labeled with reference 100 in FIG. 1) and set screws. The bone screws 110 include threading 112 that engages bone (often the head 120), a screw shaft 114 and a screw head 116 for engaging a tool. The description below will note variations and in many cases, will not repeat similar items where certain differences would be clear to one of ordinary skill in the art.

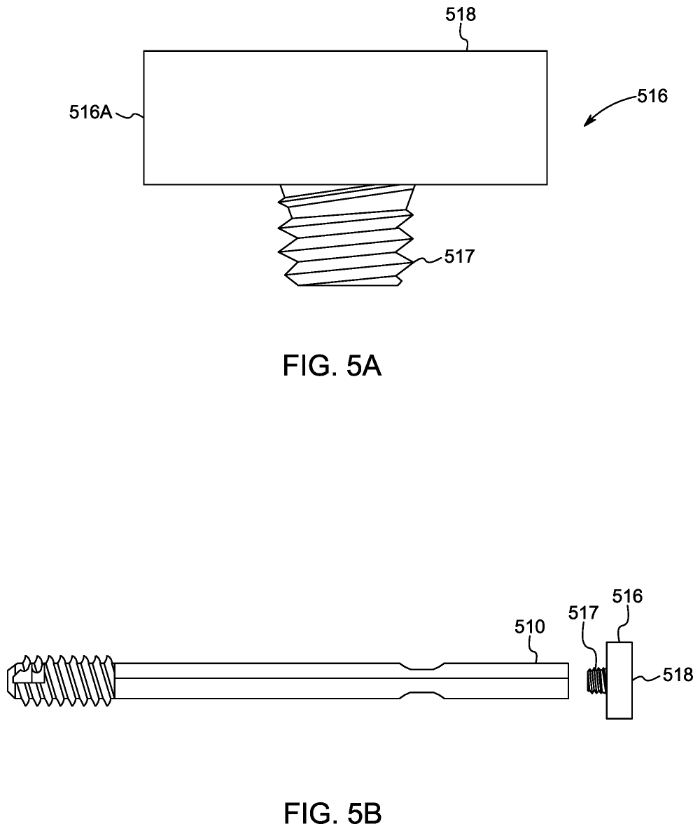

FIGS. 5A and 5B show a threaded end cap head 516 on the main screw 510. In this embodiment, a surgeon inserts the main screw 510 into the bone and then threads an independent separate head 516 (with a diameter larger than that of the main screw 510) into the main screw 510 by engaging head screws 517 and interiorly threaded main screws (not shown). The threading not only engages the screw head 516 and main screw 510 to one another, but it also pulls the main screw 510 “backwards,” creating compression across the fracture site. The exterior surface of the head 516a may be smooth or with external threads. If there is external threading on head surface 516a, a surgeon could seat the head into lateral cortical bone, increasing screw purchase, thus preventing backout. It should be understood that the screw head 516 would be fitted with a tool engagement portion on its top side 518.

It is possible to have no head on the screws, which allows seating of a proximal screw end flush with the lateral bone cortex. This prevents compression of fracture gap by a user while contacting strong cortical bone including subchondral and lateral cortical bone, while not relying on soft cancellous bone for support.

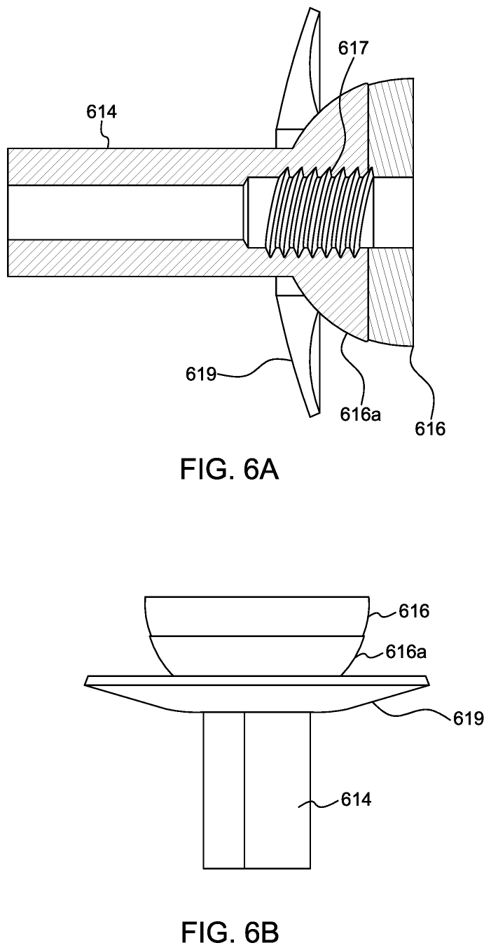

FIGS. 6A and 6B show a washer 619 or articulating head that conforms to the lateral bone surface in an embodiment similar to that discussed in FIGS. 5A and 5B, in which a screw head element 616 is removable from the main screw shaft 614, and in particular its main screw head extension 616a. The washer 619 may move relative to the lateral bone cortex during engagement. The washer may be integral with the head element 616a and or main screw shaft 614 through a snap fit engagement or other secure non-removable engagement that allows for movement of the waster 619 relative to the other parts and bone.

Alternatively, a washer may be added under the screw head on the shaft Or the head may be removable (like in FIGS. 5A-5B, and the screw head may include an integral washer. Or in a similar embodiment within a removable head like in FIGS. 5A and 5B, the head itself may articulate (with no washer).

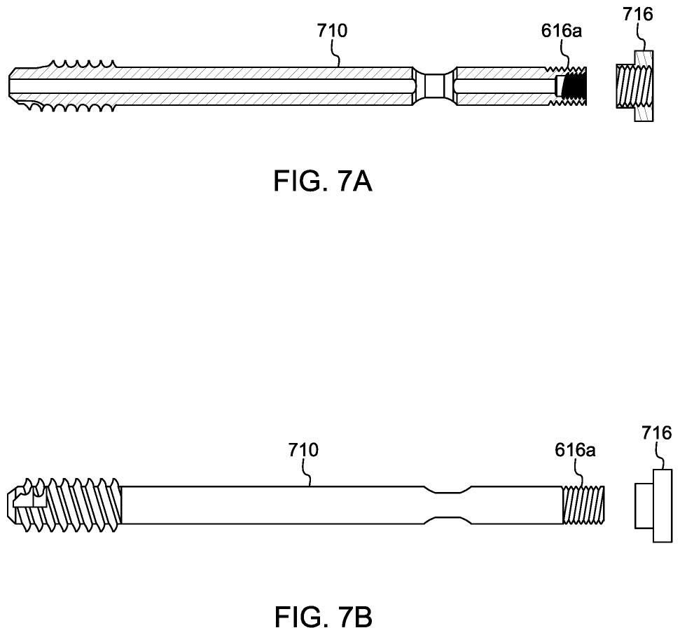

FIGS. 7A and 7B show another variation on the embodiment in FIGS. 5A and 5B in which after the surgeon inserts the main screw 710, an independent/separate head 716 is threaded into the main screw 710 (this may pull the main screw “backward”) creating compression across the fracture site. The exterior surface of the main screw end 616a may include external threads that engage the screw head 716 that is itself internally threaded. If there is external threading on the head 716, the surgeon may engage the lateral cortical bone increasing screw purchase, thus preventing backout.

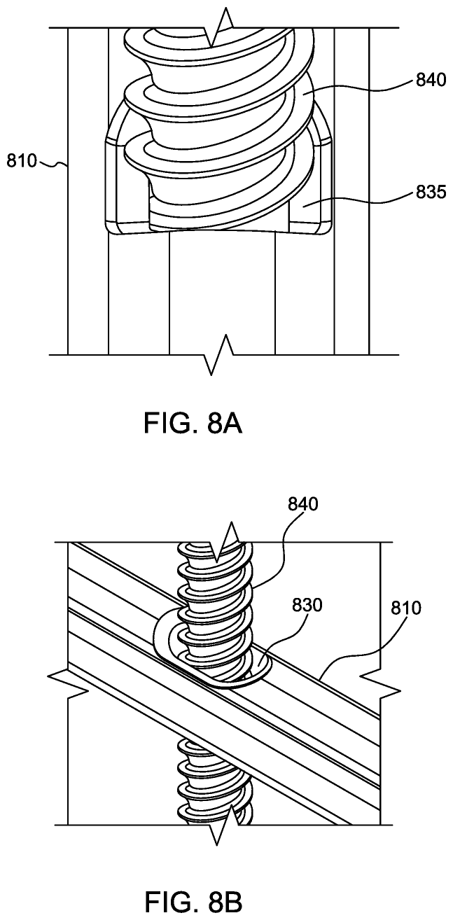

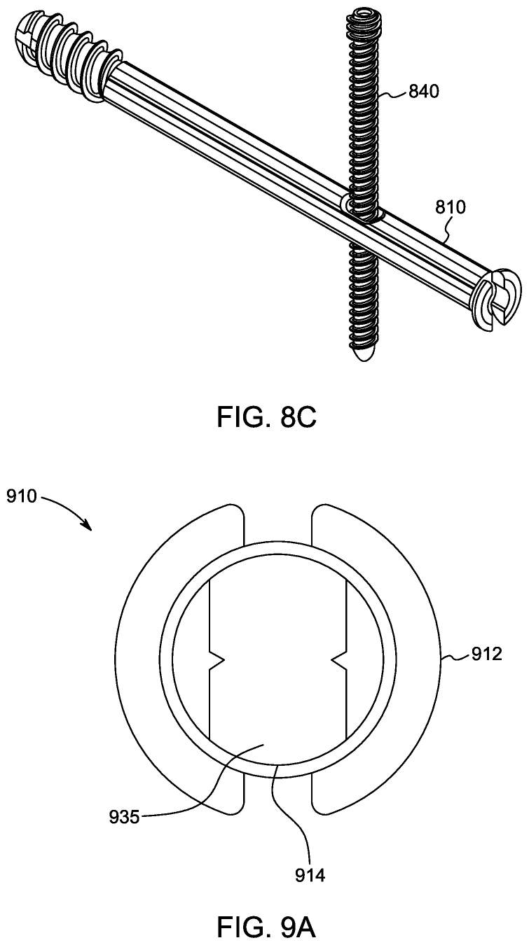

FIGS. 8A-8C show a main screw ring/shelf 835 located in its open slot 830 that engages the cross screw 840. The ring/shelf 835 around and/or inside of the edge of the clearance slot 830 allows insertion of the cross screw 840 and prevents vertical translation of main screw 810 along set screw 840. This interference fit allows for horizontal translation of the main screw 810, that is, the cross screw 840 can slide back and forth in the slot 830.

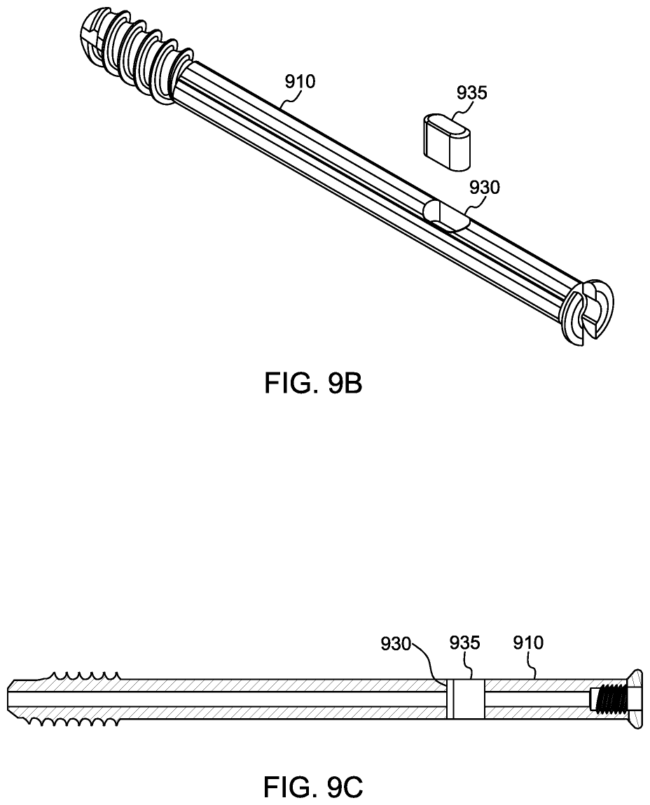

FIGS. 9A-9C show a polymer (resorbing or static) insert 935 that snaps into the main screw clearance slot 930. Before inserting main screw 910, a biocompatible material insert 935 is placed into the clearance slot 930, thus extending through channel 914 of the main screw 910. The cross screw (not shown in these FIGS. 9A-9C) is then placed through this biocompatible material insert 935, either directly or after pre-drilling. The biocompatible material can be resorbing (polymer or other material) or non-resorbing (like silicone). The resorbing material allows the screw to be temporarily fixed in one position, then secondarily allowing translation of the cross screw in the slot. Using non-resorbing material and cross screw allows it to act like a strut compressing, then rebounding.

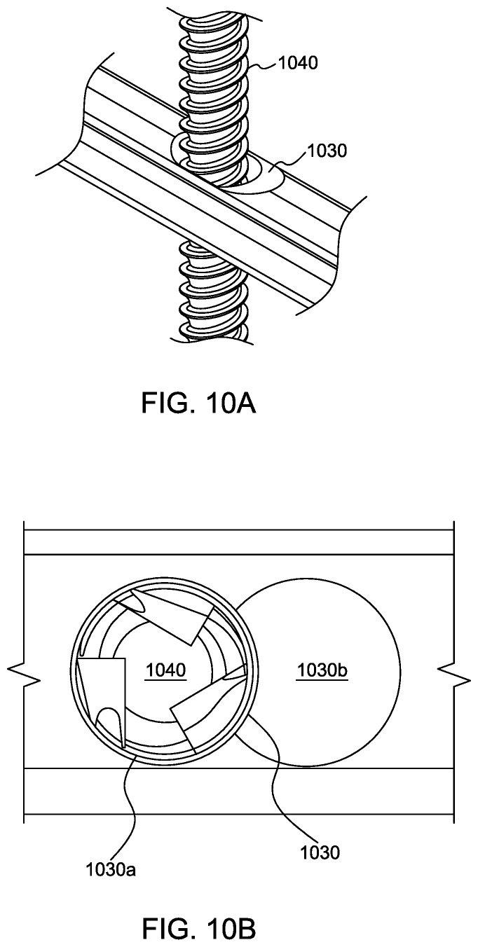

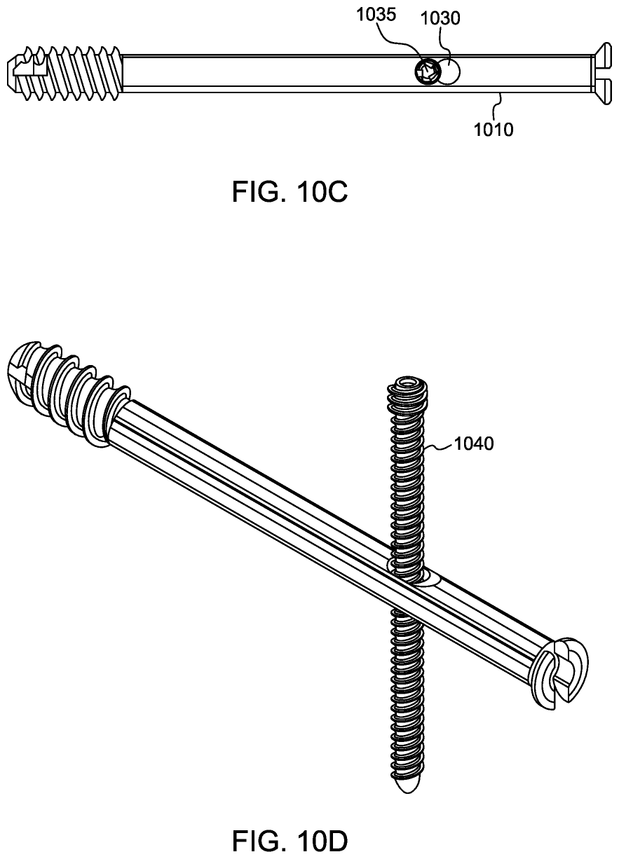

FIGS. 10A-10D show a main screw slot 1030, which may be broken into two scalloped shapes as shown. One of the scallops may be occupied by a polymer 1030b to act as a plug, while the other slot 1030a engages the cross screw 1040. The polymer insert 1030b prevents movement (fracture distraction/compression) of the main screw 1010 relative to the cross screw 1040.

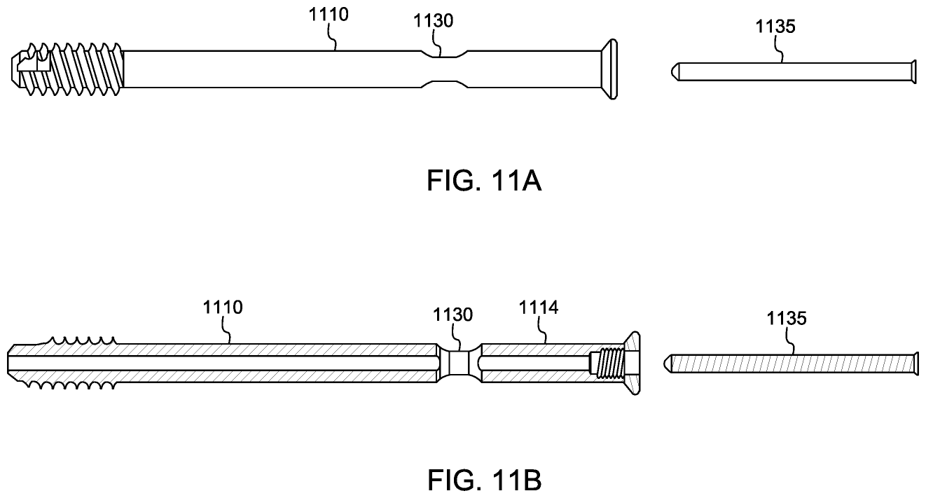

FIGS. 11A and 11B show a resorbing core 1135 that threads/snaps into an interior channel 1114 of the main screw 1110. The core 1135 is inserted into the main screw 1110 before the cross screw is drilled and/or inserted. Then, the cross screw is driven directly through (transecting) core insert 1135 that extends through the channel 1114 to the slot 1130. This core insert 1135 may temporarily inhibit vertical translation (the main screw 1110 sliding up and down the cross screw) or fracture distraction/compression (horizontal translation of cross screw sliding in slotted hole 1130 of the main screw).

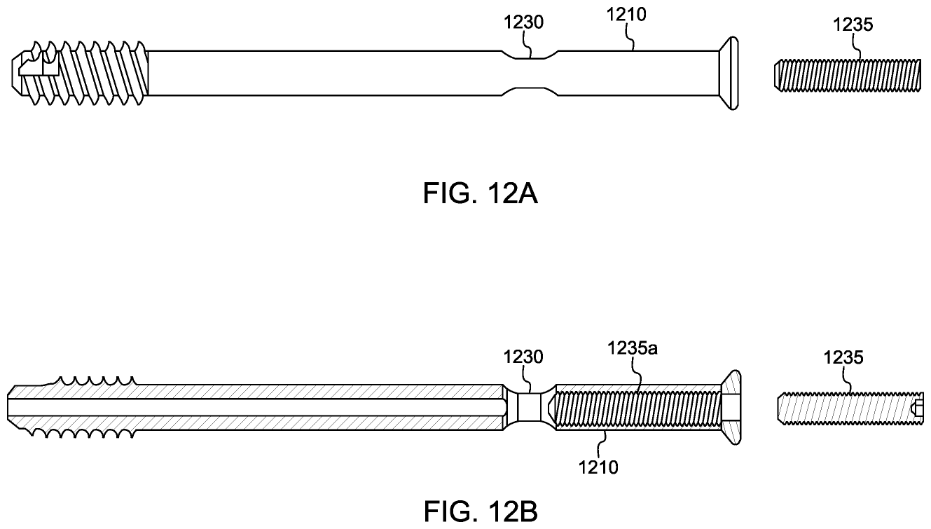

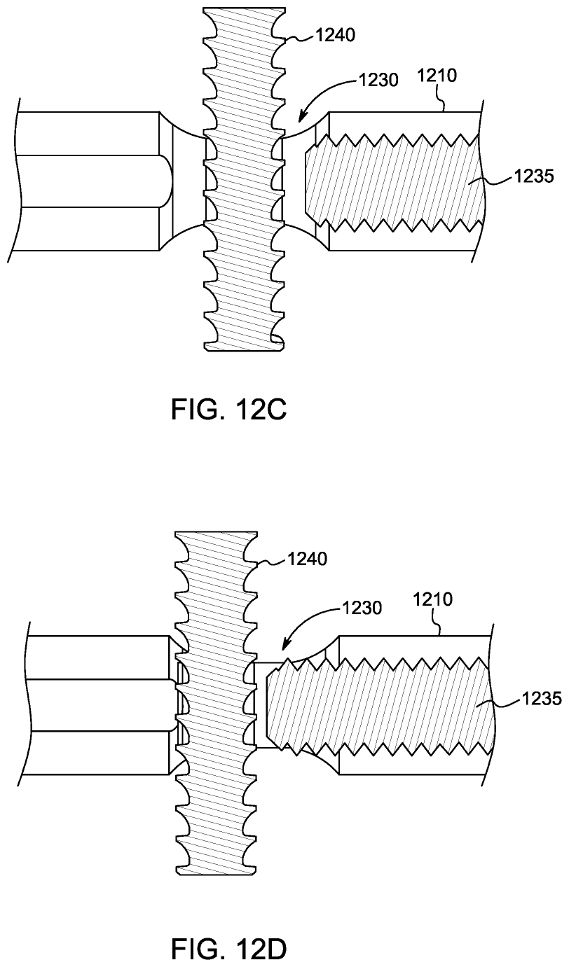

FIGS. 12A-12D show a set screw 1235 that threads/snaps into an interior of main screw 1210 to engage the cross screw 1240. The set screw 1235 may be inserted into the channel 1235a after the cross screw 1240 is placed. As shown in the progression from FIG. 12C to 12D, the set screw 1235 engages the cross screw 1240 so that the main screw 1210 and cross screw 1240 do not move relative to one another, or so that they only move relative to one another within a predetermined amount of movement The set screw 1235 may inhibit vertical translation (main screw sliding up and down cross screw) or fracture distraction/compression (horizontal translation of cross screw sliding in slotted hole 1230 of the main screw 1210). The set screw 1235 can be a metal, resorbing, or non-resorbing material.

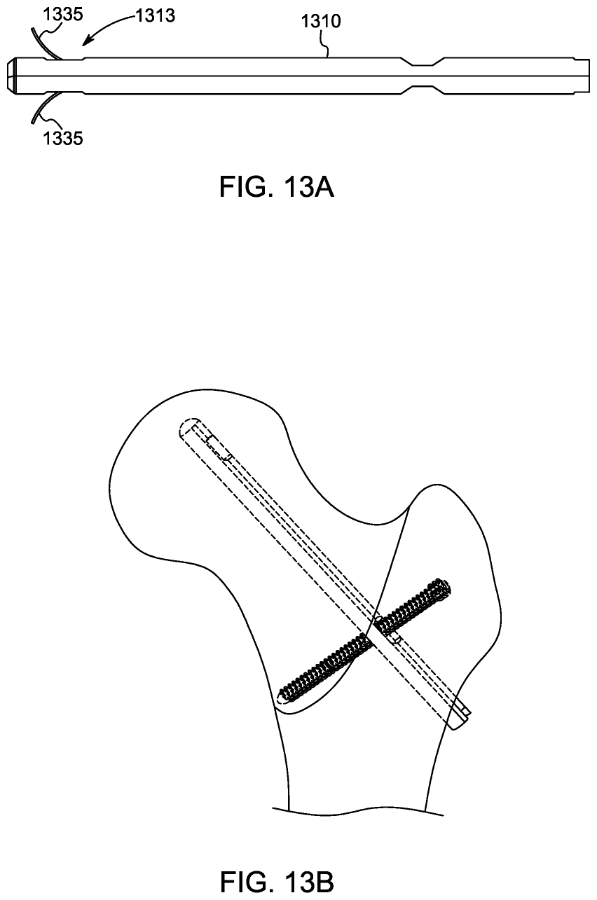

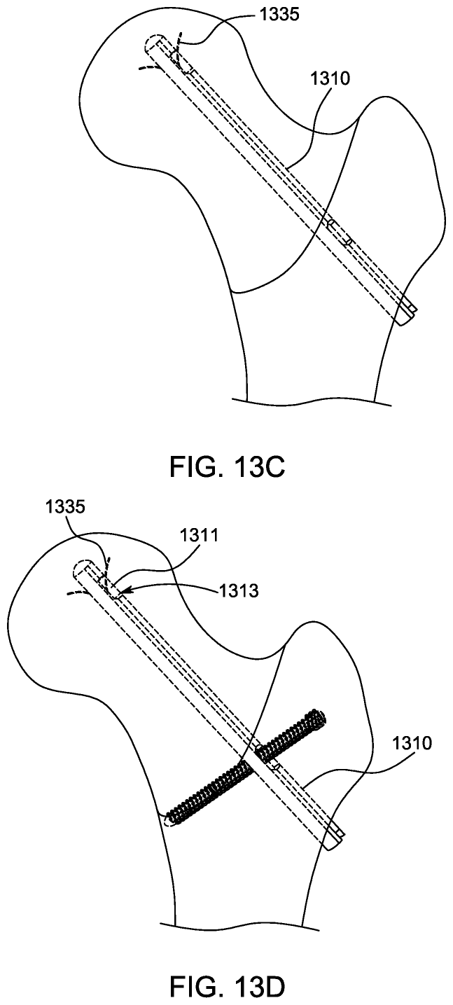

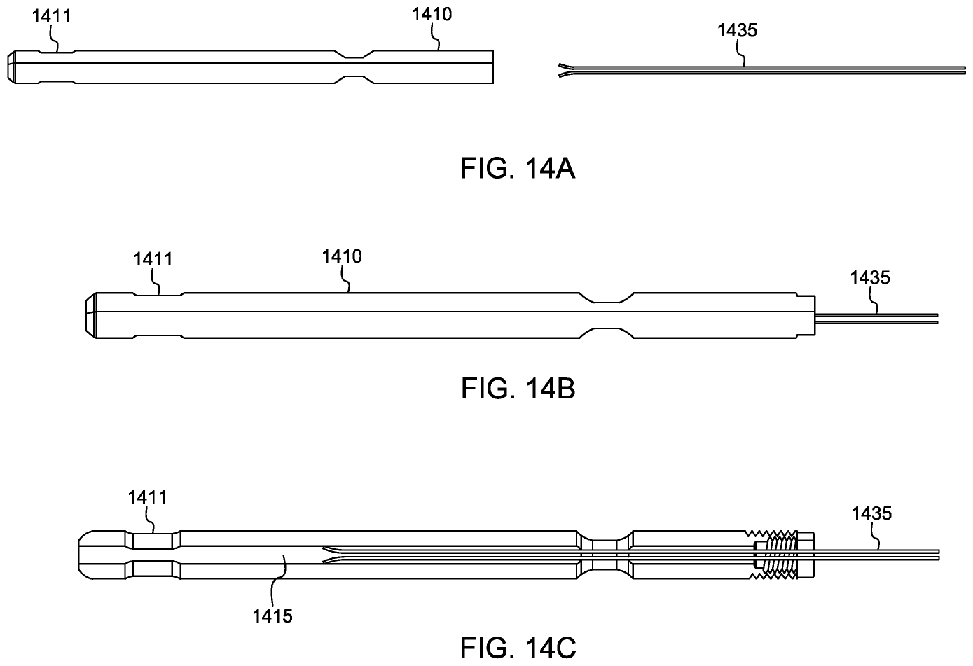

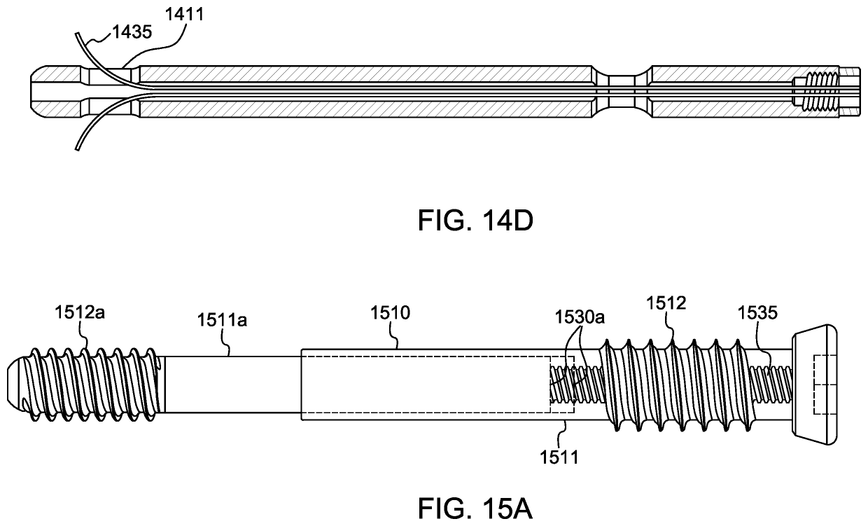

FIGS. 13A-D show extendible hooks 1335 that extend from a hole 1311 in an end 1313 of the main screw 1310 to prevent its rotation and inadvertent withdrawal. The hooks 1335 may be expandible through pressure or twisting of an internal screw along a channel (see FIG. 14C channel 1415) in the main screw 1310. The hooks 1335 may be insertable into the channel for expansion from an end 1313 of the main screw.

FIGS. 14A-14D show the hook 1435 extending through a channel 1415 that extends along the main screw 1410 length to an opening 1411. The opening 1411 may be at a distal end as shown or proximal end (between the slot and screw head area) of the main screw 1410. In practice, the hooks may be slid into an opening in the screw head end, down the channel 1415, out of the opening 1411, and into bone.

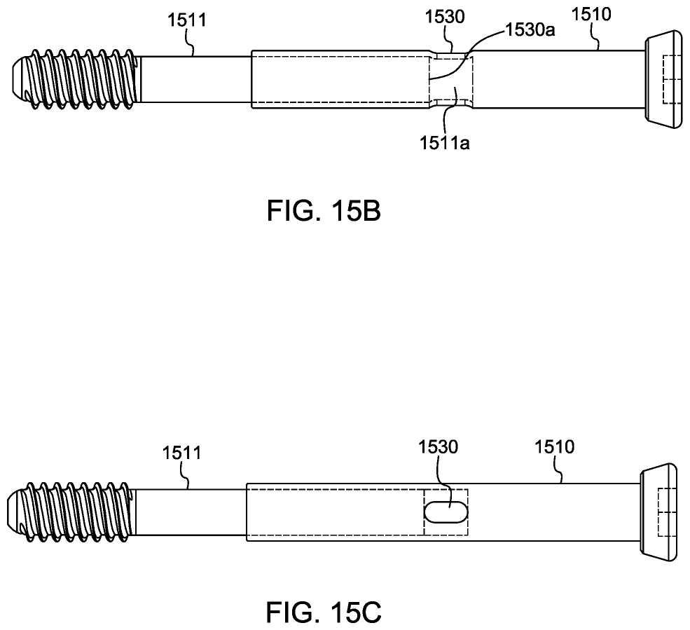

FIGS. 15A to 15C show variations of a telescoping main screw 1510. The main screw 1510 comprises two portions that move relative to one another, a main portion 1511 and a movable portion 1511a, wherein the movable portion 1511a moves within the main portion 1511. As shown in an embodiment in FIG. 15A, the main portion 1511 has proximal threading 1512 and the movable portion has distal threading 1512a. In use, a set screw 1535 within the main portion 1511 drives the movable portion surface 1530a to a desired distance and the screw as a whole (both portions) can be driven into and through the fracture. Once the proximal threads 1512a engage bone, the set screw 1535 can be reversed some distance, creating a gap between the movable portion surface 1530a and the set screw, and the proximal threading 1512 can be turned deeper into the bone, drawing the fracture closed. In this way, the surgeon can ensure that the screw will not back out because of multiple threaded engagements, and that the fracture is securely set

The internal mechanism can be fixed or adjustable to control the amount of moveable portion movement. The internal mechanism can be metal, elastomeric, or bioabsorbable. A metal mechanism maintains a fixed position/fixed movement of the movable piston. An elastomeric mechanism would allow for piston compression against some resistance. An elastomeric mechanism would also allow for a potential piston rebound against weight-bearing forces. A bioabsorbable mechanism would prevent/control movement of the piston, then allow greater compression after resorption.

In the telescoping variation shown in FIGS. 15B and 15C, there may be no set screw when compared to FIG. 15A, but there is a slot 1530 similar to the slots shown before where it receives a cross screw. But in this main portion 1511/moveable portion 1511a embodiment in FIGS. 15B and 15C, the movable surface 1530a engages the cross screw as it passes through the slot 1530.

A further variation could use threading on both the main portion 1510 and moveable portion 1511, where the main portion has a slot 1530. This would combine the embodiments in FIGS. 15A, 15B, and 15C.

Any of the screws may also be a nonthreaded peg.

The materials for the screws or pegs may include metallic components such as, but not limited to titanium, stainless steel, cobalt chrome, and carbon. Polymer materials may include, but are not limited to PLGA, PLLA, PGA, PLDLA, PEEK, polymers containing citrates, and other admixtures. Other materials may include calcium phosphates, hydroxyapatite, calcium citrates, silica, magnesium, calcium, and other natural minerals found in bone. Any of the materials may also be resorbing.

While the invention has been described with reference to the embodiments above, a person of ordinary skill in the art would understand that various changes or modifications may be made thereto without departing from the scope of the claims.

We claim:

1. A device for securing a fractured bone together comprising:
a main screw configured to be driven through a bone fracture, the main screw engageable to a removable screw head.

2. The device of claim 1, wherein the removable screw head has a larger circumference than the main screw.

3. The device of claim 2, wherein the screw head includes a tool engagement portion.

4. The device of claim 3, wherein the main screw comprises internal threading that engages external threads on the removable screw head.

5. The device of claim 3, wherein the main screw comprises external threading and the screw head comprises internal threading.

6. The device of claim 5, wherein the main screw external threading that engaged the screw head is configured to also engage bone.

7. A device for securing a fractured bone together comprising:
a main screw configured to be driven through a bone fracture, the main screw including a slot configured to engage a cross screw therethrough, wherein the slot includes a system that prevents lateral movement of the main screw relative to the cross screw.

8. The device of claim 7, wherein the system includes a shelf located within the slot configured to engage the cross screw.

9. The device of claim 7, wherein the system includes a polymer insert within the slot configured to engage the cross screw.

10. The device of claim 7, wherein the slot comprises two scallops, wherein one scallop is configured to engage the cross screw and one scallop receives a polymer that engages the cross screw to prevent lateral movement of the cross screw relative to the main screw.

11. The device of claim 7, wherein the main screw includes a channel therein that extends from a proximal end of the main screw to the slot, wherein the system includes an insert that extends through the channel and into the slot, wherein the insert is configured to engage the cross screw.

12. The device of claim 7, wherein the main screw includes a channel therein that extends from a proximal end of the main screw to the slot, wherein the system includes a set screw that extends through the channel and into the slot, wherein the set screw is configured to engage the set screw.

13. A device for securing a fractured bone together comprises a main screw, comprising a main portion and a movable portion, wherein the movable portion moves within the main portion, wherein one of the main portion or movable portion comprises threads configured to engage bone.

14. The device of claim 13, wherein the main portion includes internal threading for receiving a set screw that drives movable portion to extend a full length of the main screw.

15. The device of claim 14, wherein the movable portion includes threads configured to engage bone.

16. The device of claim 15, wherein the main portion includes threads configured to engage bone.

17. The device of claim 13, wherein the main portion comprises a slot therethrough, wherein when a cross screw is engaged within the slot, the cross screw engages the movable portion and prevents the movable portion from retreating further within the main portion.

18. A device for securing a fractured bone together comprising a main peg comprising a channel therethrough that extends between a hole in a proximal end thereof to a hook hole, and a hook that: extends from the channel, through the hook hole, and is configured to engage bone.

19. The device of claim 18, further comprising a slot in the main peg configured to receive a cross screw that inhibits movement between the main screw and cross screw when the peg is driven into bone.

20. The device of claim 18, wherein the peg comprises external threads configured to engage bone.
